(19) United States
(12) Patent Application Publication
(10) Pub. No.: US 2023/0404860 A1
(43) Pub. Date: Dec. 21, 2023

(54) METHOD, SYSTEM, AND COMPOSITION FOR COLORING DENTAL CERAMICS

(71) Applicant: James R. Glidewell Dental Ceramics, Inc., Newport Beach, CA (US)

(72) Inventors: Yan Yang, Irvine, CA (US); Sreeram Balasubramanian, Irvine, CA (US); Anna Luu, Santa Ana, CA (US)

(73) Assignee: James R. Glidewell Dental Ceramics, Inc., Newport Beach, CA (US)

(21) Appl. No.: 18/338,011

(22) Filed: Jun. 20, 2023

Related U.S. Application Data
(60) Provisional application No. 63/353,932, filed on Jun. 21, 2022.

(51) Int. Cl. A61K 6/20; A61K 6/78; A61K 6/824

(52) U.S. Cl. CPC A61K 6/20; A61K 6/78; A61K 6/824

(57) ABSTRACT

A method for coloring a porous ceramic dental body that includes:
applying at least one coating of a liquid coloring composition to at least a portion of a surface area of the porous ceramic dental body, the liquid coloring composition comprising:
(a) at least one opaquing agent comprising a material comprising Zn, Al, Si, or a combination thereof;
(b) at least one coloring agent comprising a material comprising Fe, Ni, Cu, Mn, Co, Cr, Mo, Pr, Nd, Er, Ce, Tb, or a combination thereof,
(c) at least one wetting agent; and
(d) at least one solvent; and
sintering the coated porous ceramic dental body to obtain a fully sintered ceramic body having a density that is at least 98% of the theoretical density.

This application claims the benefit of and priority to U.S. Provisional Patent Appl. No. 63/353,932, filed Jun. 21, 2022, which is incorporated herein by reference in its entirety.

### BACKGROUND

Dental ceramic materials have been widely used for restorations because of useful properties, including esthetics, chemical resistance, mechanical stability, and biocompatibility. Good esthetics (i.e., a natural-looking appearance) play a critical role for patient selection of dental restorations. A significant esthetic consideration is shading of dental restorations. Currently, coloring solutions in the market have limited shading effects. Limitations include: (1) several coloring solutions are non-homogeneous liquid systems that include precipitants (e.g., Zirkonzahn Colour Liquid Prettau® Aquarell); (2) current coloring solutions provide minimal color and/or shade when applied to dental ceramics that have high translucency; (3) current coloring solutions cannot mimic the natural tooth opacity transition from gingival area to incisal area; and (4) do not provide sufficient dental ceramic penetration during the coloring process, resulting in challenging dental restoration preparation.

### SUMMARY

Disclosed herein is a method for coloring a porous ceramic dental body comprising:
applying at least one coating of a liquid coloring composition to at least a portion of a surface area of the porous ceramic dental body, the liquid coloring composition comprising:
(a) at least one opaquing agent comprising a material comprising Zn, Al, Si, or a combination thereof;
(b) at least one coloring agent comprising a material comprising Fe, Ni, Cu, Mn, Co, Cr, Mo, Pr, Nd, Er, Ce, Tb, or a combination thereof,
(c) at least one wetting agent; and
(d) at least one solvent; and
sintering the coated porous ceramic dental body to obtain a fully sintered ceramic body having a density that is at least 98% of the theoretical density.

Also disclosed herein is a method for coloring a porous ceramic dental body comprising:
applying at least one coating of a liquid coloring composition to at least a portion of a surface area of the porous ceramic dental body, the liquid coloring composition comprising:
(a) 15 wt % to 20 wt % of a first coloring agent comprising Fe, based on the total weight of the composition;
(b) 0.5 wt % to 1 wt % of a second coloring agent comprising Ni, based on the total weight of the composition;
(c) at least one wetting agent; and
(d) at least one solvent; and
sintering the coated porous ceramic dental body to obtain a fully sintered ceramic body having a density that is at least 98% of the theoretical density.

Also disclosed herein is a method for coloring a porous ceramic dental body comprising:
applying at least one coating of a liquid coloring composition to at least a portion of a surface area of the porous ceramic dental body, the liquid coloring composition comprising:
(a) 5 wt % to 10 wt % of a coloring agent comprising Fe, based on the total weight of the composition;
(b) at least one wetting agent; and
(c) at least one solvent; and
sintering the coated porous ceramic dental body to obtain a fully sintered ceramic body having a density that is at least 98% of the theoretical density.

Also disclosed herein is a method for coloring a porous ceramic dental body comprising:
applying at least one coating of a liquid coloring composition to at least a portion of a surface area of the porous ceramic dental body, the liquid coloring composition comprising:
(a) 0.01 wt % to 0.1 wt % of a coloring agent comprising Mn, based on the total weight of the composition;
(b) at least one wetting agent; and
(c) at least one solvent; and
sintering the coated porous ceramic dental body to obtain a fully sintered ceramic body having a density that is at least 98% of the theoretical density.

Also disclosed herein is a method for coloring a porous ceramic dental body comprising:
applying at least one coating of a liquid coloring composition to at least a portion of a surface area of the porous ceramic dental body, the liquid coloring composition comprising:
(a) 1 wt % to 5 wt % of a coloring agent comprising Cu, based on the total weight of the composition;
(b) at least one wetting agent; and
(c) at least one solvent; and
sintering the coated porous ceramic dental body to obtain a fully sintered ceramic body having a density that is at least 98% of the theoretical density.

Also disclosed herein is a method for coloring a porous ceramic dental body comprising:
applying at least one coating of a liquid coloring composition to at least a portion of a surface area of the porous ceramic dental body, the liquid coloring composition comprising:
(a) 10 wt % to 15 wt % of a first coloring agent comprising Fe, based on the total weight of the composition;
(b) 10 wt % to 15 wt % of a second coloring agent comprising Ni, based on the total weight of the composition;
(c) at least one wetting agent; and
(d) at least one solvent; and
sintering the coated porous ceramic dental body to obtain a fully sintered ceramic body having a density that is at least 98% of the theoretical density.

Also disclosed herein is a method for coloring a porous ceramic dental body comprising:
applying at least one coating of a liquid coloring composition to at least a portion of a surface area of the porous ceramic dental body, the liquid coloring composition comprising:
(a) 45 wt % to 60 wt % of a coloring agent comprising Er, based on the total weight of the composition;
(b) at least one wetting agent; and
(c) at least one solvent; and
sintering the coated porous ceramic dental body to obtain a fully sintered ceramic body having a density that is at least 98% of the theoretical density.

Also disclosed herein is a method for coloring a porous ceramic dental body comprising:
applying at least one coating of a liquid coloring composition to at least a portion of a surface area of the porous ceramic dental body, the liquid coloring composition comprising:
(a) 45 wt % to 60 wt % of a first coloring agent comprising Er, based on the total weight of the composition;
(b) 0.5 wt % to 1 wt % of a second coloring agent comprising Co, based on the total weight of the composition;
(c) at least one wetting agent; and
(d) at least one solvent; and
sintering the coated porous ceramic dental body to obtain a fully sintered ceramic body having a density that is at least 98% of the theoretical density.

Also disclosed herein is a method for coloring a porous ceramic dental body comprising:
applying at least one coating of a liquid coloring composition to at least a portion of a surface area of the porous ceramic dental body, the liquid coloring composition comprising:
(a) 45 wt % to 60 wt % of a first coloring agent comprising Er, based on the total weight of the composition;
(b) 1 wt % to 3 wt % of a second coloring agent comprising Cu, based on the total weight of the composition;
(c) at least one wetting agent; and
(d) at least one solvent; and
sintering the coated porous ceramic dental body to obtain a fully sintered ceramic body having a density that is at least 98% of the theoretical density.

Also disclosed herein is a liquid coloring composition comprising:
(a) a Zn(NO₃)₂·6H₂O opaquing agent;
(b) an AlCl₃·6H₂O opaquing agent;
(c) an Fe(NO₃)₃·9H₂O coloring agent;
(d) a Ni(NO₃)₂·6H₂O coloring agent;
(e) polypropylene glycol; and
(g) at least one solvent.

Additionally disclosed herein is a liquid coloring composition comprising:
(a) 9 wt % to 11 wt % of a Zn-containing opaquing agent, or 22 wt % to 25 wt % of a Zn-containing opaquing agent;
(b) 3 wt % to 4 wt % of an Al-containing opaquing agent;
(c) 0.5 wt % to 10 wt % of an Fe-containing coloring agent;
(d) 0.3 wt % to 2 wt % of a Ni-containing coloring agent; and
(e) polypropylene glycol.

Also disclosed herein is a coloring system kit for coloring a porous ceramic dental body comprising at least 20 unique liquid coloring compositions, wherein each coloring composition comprises:
(a) a Zn(NO₃)₂·6H₂O opaquing agent;
(b) an AlCl₃·6H₂O opaquing agent;
(c) an Fe(NO₃)₃·9H₂O coloring agent;
(d) a Ni(NO₃)₂·6H₂O coloring agent;
(e) polypropylene glycol; and
(g) at least one solvent.

Further disclosed herein is a coloring system kit for coloring a porous ceramic dental body comprising a plurality of unique liquid coloring compositions, wherein
(i) a first coloring composition comprises:
   (a) a Zn(NO₃)₂·6H₂O opaquing agent;
   (b) an AlCl₃·6H₂O opaquing agent;
   (c) an Fe(NO₃)₃·9H₂O coloring agent;
   (d) a Ni(NO₃)₂·6H₂O coloring agent;
   (e) polypropylene glycol; and
   (g) at least one solvent;
(ii) a second coloring composition comprises:
   (a) 15 wt % to 20 wt % of a first coloring agent comprising Fe, based on the total weight of the composition;
   (b) 0.5 wt % to 1.0 wt % of a second coloring agent comprising Ni, based on the total weight of the composition;
   (c) polypropylene glycol; and
   (d) at least one solvent;
(iii) a third coloring composition comprises:
   (a) 8 wt % to 10 wt % of a coloring agent comprising Fe, based on the total weight of the composition;
   (b) polypropylene glycol; and
   (c) at least one solvent;
(iv) a fourth coloring composition comprises:
   (a) 0.03 wt % to 0.06 wt % of a coloring agent comprising Mn, based on the total weight of the composition;
   (b) polypropylene glycol; and
   (c) at least one solvent;
(v) a fifth coloring composition comprises:
   (a) 1 wt % to 3 wt % of a coloring agent comprising Cu, based on the total weight of the composition;
   (b) polypropylene glycol; and
   (c) at least one solvent;
(vi) a sixth coloring composition comprises:
   (a) 12 wt % to 13 wt % of a first coloring agent comprising Fe, based on the total weight of the composition;
   (b) 12 wt % to 13 wt % of a second coloring agent comprising Ni, based on the total weight of the composition;
   (c) polypropylene glycol; and
   (d) at least one solvent; and
(vii) a seventh coloring composition comprises:
   (a) 45 wt % to 60 wt % of a first coloring agent comprising Er, based on the total weight of the composition;
   (b) polypropylene glycol; and
   (c) at least one solvent;
(viii) an eighth coloring composition comprises:
   (a) 45 wt % to 60 wt % of a first coloring agent comprising Er, based on the total weight of the composition;
   (b) 1 wt % to 3 wt % of a second coloring agent comprising Cu, based on the total weight of the composition;
   (c) polypropylene glycol; and
   (d) at least one solvent; and
(ix) a ninth coloring composition comprises:
   (a) 45 wt % to 60 wt % of a first coloring agent comprising Er, based on the total weight of the composition;
   (b) 0.5 wt % to 1 wt % of a second coloring agent comprising Co, based on the total weight of the composition;
   (c) polypropylene glycol; and
   (d) at least one solvent.

The foregoing will become more apparent from the following detailed description, which proceeds with reference to the accompanying figures.

### BRIEF DESCRIPTION OF THE DRAWINGS

FIG. 1 depicts an anterior dental restoration having facial surface locations identified as A, B, C, D and E.

FIG. 2 shows the location of a cross-section for measuring L and b values on a dental bridge pontic.

FIGS. 3 and 4 are graphs showing b values and L values, respectively.

FIG. 5 are photographs comparing precipitation of a comparative coloring composition and the inventive liquid coloring composition as disclosed herein.

### DETAILED DESCRIPTION

Disclosed herein are methods and liquid coloring compositions for shading ceramic dental bodies prior to sintering. The coloring compositions provide for a coloring system that can offer multiple shades to produce a better shade match in the body part and the occlusal part of a dental restoration. For example, the coloring system can include at least 20, more particularly 20 to 30, and most particularly 26, unique liquid coloring compositions that produce a better shade match for customizing dental restorations with improved esthetic properties. In certain embodiments, the coloring compositions provide more chroma when applied to high translucency dental restorations.

The liquid coloring composition is applied to a dental ceramic body prior to sintering. The liquid coloring composition may be applied to the full or partial surface area of the ceramic body. In certain embodiments, the liquid coloring composition is applied to the full surface area of the ceramic body. The dental ceramic body may be a dental prostheses in the form of a crown, veneer, single or multi-unit bridge, implant-supported partial or full-arch denture, and the like, that attach to the support structure of a patient, such as an implant abutment or natural tooth preparation.

The liquid coloring composition includes at least one coloring agent, at least one opaquing agent, at least one wetting agent, and at least one solvent.

The coloring agent(s) can produce dentally acceptable shade effects on dental materials, even with high translucency. For example, the coloring agent(s) can achieve a final color in the sintered yttria-stabilized zirconia ceramic material that matches a shade from a VITA A1-D4@ Classical Shades shade guide or VITA Bleached Shades shade guide, such as 0M1, 0M2 or 0M3 bleach shades, (available through VITA North America) when measured according to the shade match evaluation test method provided herein.

The coloring agent can be a transition metal-containing material, such as a metal complex or metallic compound, for example, a metallic salt. Illustrative transition metal-containing materials suitable for use as coloring agents include oxides or salts of one or more elements selected from Fe, Ni, Cu, Mn, Co, Cr, and Mo, or a combination thereof. Metal-containing materials may comprise an anion such as acetate, oxalate, sulfate, carbonate, halide (e.g., chloride or fluoride), nitrate, phosphate or citrate. In certain embodiments, the coloring agent is a hydrate of the metallic salt.

The coloring agent can be a rare earth metal-containing material, such as a rare earth metal complex or rare earth metallic compound, for example, a rare earth metallic salt. Illustrative rare earth metal-containing materials suitable for use as coloring agents include oxides or salts of one or more elements selected from Pr, Nd, Er, Ce, and Tb, or a combination thereof. Rare earth element-containing materials may include an anion such as acetate, oxalate, sulfate, carbonate, halide (e.g., chloride or fluoride), nitrate, phosphate or citrate.

In one embodiment, a coloring agent in the form of a metallic salt is selected that is soluble in an aqueous liquid coloring composition. The coloring agent may be added to the coloring composition in the form of a solid or a liquid.

Illustrative coloring agents include Fe(NO₃)₃.9H₂O, Ni(NO₃)₂·6H₂O, CuCl₂·2H₂O, MnSO₄·H₂O, Er(NO₃)₃·6H₂O, CoCl₂·6H₂O, Nd(NO₃)₃·6H₂O, Cr(NO₃)₃·9H₂O, and Tb(NO₃)₃·6H₂O.

The coloring composition may include 0.03 wt % to 70 wt %, more particularly 2 wt % to 60 wt %, and most particularly 15 wt % to 30 wt %, most particularly 45 wt % to 60 wt % of the coloring agent, or a mixture of coloring agents, based on the total weight of the coloring composition. In certain embodiments, the coloring composition includes 0.3 wt % to 25 wt %, more particularly 2 wt % to 13 wt %, and most particularly 5 wt % to 10 wt %, of an Fe-containing coloring agent (e.g., Fe(NO₃)₃·9H₂O). In certain embodiments, the coloring composition includes 0.3 wt % to 25 wt %, most particularly 0.3 wt % to 2 wt % and 10 wt % to 15 wt %, of a Ni-containing coloring agent (e.g., Ni(NO₃)₂·6H₂O). In certain embodiments, the coloring composition includes 0.01 wt % to 10 wt %, more particularly 0.05 wt % to 0.5 wt %, and 1 wt % to 3 wt %, of a Cu-containing coloring agent (e.g., CuCl₂·2H₂O). In certain embodiments, the coloring composition includes 0.01 wt % to 3 wt %, more particularly 0.03 wt % to 0.1 wt %, of a Mn-containing coloring agent (e.g., MnSO₄·H₂O).

Another embodiment of a liquid coloring composition includes 5 wt % to 20 wt % (more particularly 18 wt % to 20 wt %) of a first coloring agent comprising Fe (e.g., Fe(NO₃)₃·9H₂O), 0.5 wt % to 15 wt % (more particularly 0.8 wt % to 1.0 wt %) of a second coloring agent comprising Ni (e.g., Ni(NO₃)₂·6H₂O), at least one wetting agent, and at least one solvent. In certain embodiments, the Fe-containing coloring agent and the Ni-containing coloring agent are the only coloring agents present in the composition. This composition can provide an orange shade coloring composition.

Another embodiment of a liquid coloring composition includes 5 wt % to 10 wt % (more particularly 9 wt % to 10 wt %) of a coloring agent comprising Fe (e.g., Fe(NO₃)₃·9H₂O), at least one wetting agent, and at least one solvent. In certain embodiments, the Fe-containing coloring agent is the only coloring agent present in the composition. This composition can provide a yellow shade coloring composition.

Another embodiment of a liquid coloring composition includes 0.03 wt % to 0.06 wt % (more particularly 0.04 wt % to 0.06 wt %) of a coloring agent comprising Mn (e.g., MnSO₄·H₂O), at least one wetting agent, at least one solvent. In certain embodiments, the Mn-containing coloring agent is the only coloring agent present in the composition. This composition can provide a grey shade coloring composition.

Another embodiment of a liquid coloring composition includes 1 wt % to 5 wt % (more particularly 2 wt % to 3 wt %) of a coloring agent comprising Cu (e.g., CuCl₂·2H₂O), at least one wetting agent, and at least one solvent. In certain embodiments, the Cu-containing coloring agent is the only coloring agent present in the composition. This composition can provide a green shade coloring composition.

Another embodiment of a liquid coloring composition includes 10 wt % to 15 wt % (more particularly 12 wt % to 13 wt %) of a first coloring agent comprising Fe (e.g., Fe(NO₃)₃·9H₂O), 10 wt % to 15 wt % (more particularly 12 wt % to 13 wt %) of a second coloring agent comprising Ni (e.g., Ni(NO₃)₂·6H₂O), at least one wetting agent, and at least one solvent. In certain embodiments, the Fe-containing coloring agent and the Ni-containing coloring agent are the only coloring agents present in the composition. This composition can provide a brown shade coloring composition.

Another embodiment of a liquid coloring composition includes 45 wt % to 60 wt % (more particularly 55 wt % to 60 wt %) of a first coloring agent comprising Er (e.g., Er(NO₃)₃·6H₂O), at least one wetting agent, and at least one solvent. In certain embodiments, the Er-containing coloring agent is the only coloring agent present in the composition. This composition can provide a pink shade coloring composition.

Another embodiment of a liquid coloring composition includes 45 wt % to 60 wt % (more particularly 45 wt % to 50 wt %) of a first coloring agent comprising Er (e.g., Er(NO₃)₃·6H₂O), comprising 0.5 wt % to 1 wt % (more particularly 0.8 wt % to 1 wt %) of a second coloring agent comprising Co (e.g., CoCl₂·6H₂O), at least one wetting agent, and at least one solvent. In certain embodiments, the Er-containing coloring agent and the Co-containing coloring agent are the only coloring agents present in the composition. This composition can provide a purple shade coloring composition.

Another embodiment of a liquid coloring composition includes 50 wt % to 60 wt % (more particularly 53 wt % to 56 wt %) of a first coloring agent comprising Er (e.g., Er(NO₃)₃·6H₂O), comprising 1 wt % to 3 wt % (more particularly 1 wt % to 2 wt %) of a second coloring agent comprising Cu (e.g., CuCl₂·2H₂O), at least one wetting agent, and at least one solvent. In certain embodiments, the Er-containing coloring agent and the Cu-containing coloring agent are the only coloring agents present in the composition. This composition can provide a blue shade coloring composition.

The coloring composition may include 0.05 g/L to 500 g/L, more particularly 0.1 g/L to 250 g/L, and most particularly 20 g/L to 80 g/L, or 150 g/L to 210 g/L, of the metal ion of the coloring agent, or a mixture of coloring agents.

The opaquing agent(s) can modify the L value of dental materials and create opaque effects on the body part of the dental restorations, producing more esthetic effects to better mimic the nature tooth appearance. The opaquing agent can be a material containing Zn, Al, Si, or a combination thereof. The opaquing agent may a complex or a compound, for example, a salt or an oxide. The opaquing agent may include an anion such as acetate, oxalate, sulfate, carbonate, halide (e.g., chloride or fluoride), nitrate, phosphate or citrate. In certain embodiments, the opaquing agent is a Zn-containing material or an Al-containing material. In certain embodiments, the coloring composition includes both a Zn-containing material and an Al-containing material.

In one embodiment, an opaquing agent in the form of a salt is selected that is soluble in an aqueous liquid coloring composition. The opaquing agent may be added to the coloring composition in the form of a solid or a liquid.

Illustrative opaquing agents include Zn(NO₃)₂·6H₂O, AlCl₃·6H₂O, and tetraethyl orthosilicate (TEOS).

The coloring composition may include 1 wt % to 30 wt %, more particularly 10 wt % to 15 wt %, or 25 wt % to 30 wt %, of the opaquing agent, or a mixture of opaquing agents, based on the total weight of the coloring composition. In certain embodiments, the coloring composition includes 0 wt % to 30 wt %, more particularly 5 wt % to 25 wt %, and most particularly 9 wt % to 11 wt % or 22 wt % to 25 wt %, of a Zn-containing opaquing agent (e.g., Zn(NO₃)₂·6H₂O). In certain embodiments, the coloring composition includes 0.1 wt % to 10 wt %, more particularly 1 wt % to 10 wt %, more particularly 1 wt % to 5 wt %, and most particularly 3 wt % to 4 wt %, of an Al-containing opaquing agent (e.g., AlCl₃·6H₂O). In certain embodiments, the weight ratio of Zn(NO₃)₂·6H₂O/AlCl₃·6H₂O is in the range of 1 to 10, more particularly in the range of 2 to 3 or 6.5 to 7.5. In certain embodiments, the weight ratio of metal ion Zn/Al is in the range of 3 to 15, or more particularly in the range of 3.5 to 5.5 or 13 to 14.

The coloring composition may include 0 g/L to 300 g/L, more particularly 10 g/L to 200 g/L, and even more particularly 20 g/L to 150 g/L, and most particularly 25 g/L to 80 g/L, of the opaquing agent, or a mixture of opaquing agents.

A wetting agent, such as n-propanol, glycerol, ethylene glycol, polyethylene glycol (e.g., PEG 200 or PEG 400), or polypropylene glycol (e.g., PPG 400) may be added to the liquid coloring composition to control the penetration depth of the coloring agent and/or the opaquing agent in the porous ceramic body.

The coloring composition may include 0.1 wt % to 5 wt %, more particularly 0.1 wt % to 3 wt %, and most particularly 0.5 wt % to 2 wt %, of the wetting agent, or a mixture of wetting agents, based on the total weight of the coloring composition.

The solvent can be aqueous or non-aqueous. Illustrative solvents include water, organic solvents such as ethanol alcohol, isopropyl alcohol, acetone, and mixtures thereof.

In certain embodiments, the coloring composition does not include an acid with a pH value less than 6, more particularly less than 4, and most particularly less than 3.

CIELAB color space is used to define the color difference (ΔE). In the CIELAB color space:
L represents lightness with the range of 0-100;
a represents redness-greenness of color with positive a is red and negative a is green; and
b represents yellowness-blueness of the color with the positive b is yellow and negative b is blue.
   Color difference ΔE is calculated as follows:

ΔE=(ΔL²⁺Δa*²⁺Δb*²)^{1/2}
wherein,
ΔL—lightness difference
Δb—yellowness or blueness difference, and
Δa—redness or greenness difference

The final dental restorations treated with the liquid coloring composition as disclosed herein having a ΔE of 0 to 8, more particularly 0-5, and most particularly 0-3. The final dental restorations treated with the liquid coloring composition disclosed herein having a Δb of 0 to 8, or 0 to 5, or 0 to 3, and most particularly 0 to 1.

The coloring compositions are homogenous solutions. The coloring compositions also are stable to ensure final shade consistency. In particular, the color compositions do not exhibit precipitation over a commercially useful time period (for example, up to 5 years, more particularly up to 3 years, and most particularly up to 2 years).

The liquid coloring compositions may be applied by techniques such as painting by brushing, or by dipping, or dripping, liquid coloring compositions onto the porous dental prosthesis. Liquid coloring compositions may be applied by known techniques for distributing liquid compositions onto ceramic surfaces, including coating with a marker or felt-tip pen that is loaded with the liquid mixture, or by use of a sponge.

In certain embodiments, the coloring composition can be applied to zirconia dental materials with the Y mol % in the range of 2-10 mol %.

The coloring composition can produce dentally acceptable shade effects on dental materials, even materials with high translucency. Highly translucent, sintered yttria-stabilized zirconia dental ceramics may include materials having between 40% and 80% transmittance, or between 50% and 70% transmittance, or 50% and 62% transmittance, at 700 nm when measured on a 1 mm thick sintered body.

Prior to the application of the coloring composition, bisque stage dental prostheses may be unshaded, having the color of natural zirconia materials, which may appear unnaturally white upon sintering if no colorant or staining is applied. Applying at least one coating of at least one coloring composition imparts a dentally acceptable color after sintering when applied to one or more surfaces of the dental prosthesis. Alternatively, shaded bisque stage dental prostheses may be obtained that are made from shaded ceramic powder that provides a dentally acceptable shade upon sintering. In some embodiments, a coloring composition applied to at least one surface of a shaded dental prosthesis may alter the final color or shade.

In some embodiments, a coloring composition(s) applied to the facial surface and/or internal side surfaces of a prosthesis may penetrate below the surface for a distance of at least 10000 μm, or at least 5000 μm, or at least 3000 μm, or between 0.01 μm and 3000 μm, or between 0.1 μm and 2000 μm, increasing the concentration of coloring agent and opaquing agent in this region.

Penetration of the coloring agent and/or opaquing agent into the ceramic prosthesis may be detected by energy dispersive spectroscopy (EDS) analysis of a cross-section of a dental prosthesis. Points along a line from the facial surface to the internal surface may be analyzed for the concentration of the metal element of the masking agent. In one embodiment, the concentration of metal attributable to the coloring agent may be between 0.001 wt % and 10 wt %, or between 0.004 wt % and 6 wt %, or between 0.004 wt % and 3 wt % and 4.5 wt % to 6 wt % when analyzed by EDS according to methods described herein. In one embodiment, the concentration of element attributable to the opaquing agent may be between 0.01 wt % and 5 wt %, or between 1 wt % and 3 wt %, or between 1 wt % and 1.5 wt %, and between 2.5 wt % and 3 wt %, when analyzed by EDS according to methods described herein.

Dental prostheses may comprise zirconia ceramic materials stabilized by 2 mol % to 10 mol % yttria. Yttria-stabilized zirconia ceramic material may be stabilized, for example, from 3 mol % yttria to 7.5 mol % yttria, from 4.0 mol % yttria to 7.5 mol % yttria, from 4 mol % yttria to 7 mol % yttria, from 5 mol % yttria to 8 mol % yttria, from 5 mol % yttria to 7.5 mol % yttria, from 5 mol % yttria to 7 mol % yttria.

In certain embodiments, sintered zirconia ceramic materials may be stabilized by 3 mol % to 8 mol % yttria. Starting materials for wet forming processes may include, but are not limited to, ceramic powder, dispersant, and deionized water to form ceramic slurries. Yttria-stabilized zirconia ceramic material in the slurry may comprise up to 7.5 mol % yttria, or up to 8.5 mol % yttria, for example, from 5 mol % yttria to 8.5 mol % yttria, from 5 mol % yttria to 8 mol % yttria, from 5 mol % yttria to 7.5 mol % yttria, 5 mol % yttria to 6.4 mol % yttria, from 5 mol % yttria to 5.6 mol % yttria, from 5.1 mol % yttria to 6.4 mol % yttria, from 5.2 mol % yttria to 7.5 mol % yttria, from 5.2 mol % yttria to 7.0 mol % yttria, from 5.4 mol % yttria to 7.5 mol % yttria, from 5.4 mol % yttria to 7.0 mol % yttria, from 5.5 mol % yttria to 7.5 mol % yttria, from 5.5 mol % yttria to 7 mol % yttria, from 5.5 mol % yttria to 6.9 mol % yttria, from 5.5 mol % yttria to 6 mol % yttria, from 5.5 mol % yttria to 5.9 mol % yttria, from 5.6 mol % yttria to 6.3 mol % yttria, from 5.7 mol % yttria to 6.3 mol % yttria, from 5.8 mol % yttria to 6.3 mol % yttria, from 6 mol % yttria to 8.5 mol % yttria, from 6 mol % yttria to 8 mol % yttria, from 6.0 mol % yttria to 7.5 mol % yttria, from 6 mol % yttria to 7 mol % yttria, from 6.0 mol % yttria to 6.8 mol % yttria, from 6.0 mol % yttria to 6.3 mol % yttria, from 6.2 mol % yttria to 7.5 mol % yttria, from 6.4 mol % yttria to 7.5 mol % yttria, from 7 mol % yttria to 8.5 mol % yttria, or from 7.2 mol % to 8.4 mol % yttria, to stabilize zirconia.

Zirconia ceramic material may comprise a mixture of unstabilized zirconia and stabilized zirconia ceramic materials. The term stabilized zirconia ceramic herein includes fully stabilized and partially stabilized zirconia. Specific examples include zirconia with no yttria, or yttria-stabilized zirconia including, but not limited to, commercially available yttria-stabilized zirconia, for example, from Tosoh USA, such as Tosoh TZ-3YS and Tosoh TZ-PX430. The calculated amount of yttria (e.g., yttria mol %) in zirconia ceramic material may vary from ‘nominal’ values implied by commercial nomenclature (e.g. 3YS). The mol % yttria in zirconia ceramic material may be calculated, for example, based on compositional information received from manufacturer certification.

Dental prosthetic shapes may be formed as green bodies or bisqued state bodies. Green body manufacturing methods may include dry forming processes, such as uniaxial pressing and cold isostatic pressing, and wet forming processes, including but not limited to, pressure-casting, slip-casting, filter pressing, and centrifugal casting methods. A green body manufacturing method such as a slip-casting process, may include the process steps of selecting starting materials; mixing and comminuting the starting materials to form a slurry; and casting the slurry to form a desired green body form, such as the shape of a milling blocks. Methods for making zirconia dental prosthesis materials suitable for use herein may be found in commonly owned patents and patent publications, including U.S. Pat. Nos. 9,434,651, 9,790,129, and U.S. Pat. Pub. 2018/0235847, the subject matter of each is hereby incorporated by reference in its entirety.

Yttria-stabilized zirconia ceramic materials used as starting materials to form millable blocks may, optionally, include a small amount of alumina (aluminum oxide, Al₂O₃) as an additive. For example, some commercially available yttria-stabilized zirconia ceramic material include alumina at concentrations of from 0 wt % to 2 wt %, or from 0 wt % to 0.25 wt %, such as 0.1 wt %, relative to the zirconia material. Other optional additives of the ceramic starting material may include coloring agents to obtain shaded zirconia ceramic powder that may be formed by, for example, casting or pressing into shaded ceramic blocks that have a dentally acceptable shade or pre-shade upon sintering.

Dispersants used to form ceramic suspensions or ceramic slurries to form green bodies by slip-casting manufacturing methods such as those described herein, function by promoting the dispersion and/or stability of the slurry and/or decreasing the viscosity of the slurry. Dispersion and deagglomeration may occur through electrostatic, electrosteric, or steric stabilization. Examples of suitable dispersants include nitric acid, hydrochloric acid, citric acid, diammonium citrate, triammonium citrate, polycitrate, polyethyleneimine, polyacrylic acid, polymethacrylic acid, polymethacrylate, polyethylene glycols, polyvinyl alcohol, polyvinyl pyrillidone, carbonic acid, and various polymers and salts thereof. These materials may be either purchased commercially, or prepared by known techniques. Specific examples of commercially available dispersants include Darvan® 821-A ammonium polyacrylate dispersing agent commercially available from Vanderbilt Minerals, LLC; Dolapix™ CE 64 organic dispersing agent and Dolapix™ PC 75 synthetic polyelectrolyte dispersing agent commercially available from Zschimmer & Schwarz GmbH; and Duramax™ D 3005 ceramic dispersant commercially available from Dow Chemical Company.

Zirconia ceramic and dispersant starting materials added to deionized water may be mixed to obtain a slurry. Slurries may be subjected to a comminution process for mixing, deagglomerating and/or reducing particle size of zirconia ceramic powder particles. Comminution may be performed using one or more milling process, such as attritor milling, horizontal bead milling, ultrasonic milling, or other milling or comminution process, such as high shear mixing or ultra-high shear mixing capable of reducing zirconia ceramic powder particle sizes described herein.

In one embodiment, a zirconia ceramic slurry may undergo comminution by a horizontal bead milling process. Media may comprise zirconia-based beads, for example, having a diameter of 0.4 mm. A suspension or slurry having a zirconia ceramic solids loading of about 60 wt % to about 80 wt % and a dispersant concentration from 0.002 gram dispersant/gram zirconia ceramic powder to 0.01 gram dispersant/gram zirconia ceramic powder, may be used to prepare the zirconia ceramic slurry. Milling processes may include, for example, a flow rate of 1 kg to 10 kg zirconia ceramic powder/hour, such as, approximately 6 kg zirconia ceramic powder/hour where, for example, approximately 6 kg of zirconia ceramic material is milled for approximately one hour, at a mill speed of approximately 1500 rpm to 3500 rpm, for example, approximately 2000 rpm.

In some embodiments, where commercially available zirconia ceramic is used as starting materials to prepare the ceramic slurry, the measured median particle size, or particle size distribution at D₍₅₀₎ may be about 200 nm to 600 nm, or greater than 600 nm, which includes agglomerations of particles of crystallites having a crystallite size of about 20 nm to 40 nm. As used herein, the term “measured particle size” refers to measurements obtained by a Brookhaven Instruments Corp. X-ray disk centrifuge analyzer. By processes described herein, an initial particle size distribution at, for example, a D₍₅₀₎ of about 200 nm to 600 nm, or greater than 600 nm, may be reduced to provide a zirconia ceramic material contained in a slurry having a median particle size where D₍₅₀₎ is from 100 nm to 600 nm, such as, wherein D₍₅₀₎ is from 150 nm to 350 nm, or from 220 nm to 320 nm or wherein D₍₅₀₎ is from 250 nm to 300 nm. In some embodiments, after comminution processes ceramic slurries comprise particle size distributions wherein D₍₁₀₎ is from 100 nm to 250 nm, or D₍₁₀₎ is from 120 nm to 220 nm, or D₍₁₀₎ is from 120 nm to 200 nm, and D₍₉₀₎ of zirconia particles is less than 800 nm, or D₍₉₀₎ is in the range of 250 nm to 425 nm.

By processes described herein, zirconia ceramic material may comprise an initial median particle size, for example, a D₍₅₀₎ of less than 400 nm, which upon comminution may provide a slurry comprising a zirconia ceramic material having a median particle size where D₍₅₀₎ is from 100 nm to 350 nm, such as, wherein D₍₅₀₎ is from 80 nm to 280 nm. Yttria-stabilized zirconia ceramic material comprising mixtures of two or more yttria stabilized zirconia ceramic materials each having different initial median particle sizes, may be comminuted as a mixture in a slurry by the processes described herein. Reduced particle sizes and/or narrow ranges of comminuted zirconia ceramic material, in combination with the dispersants describe above, may provide cast parts with a higher density and smaller pores that form sintered bodies having higher translucency and/or strength than those obtained by way of conventional pressing and slip-casting processes.

Zirconia ceramic slurries may be cast into a desired shape, such as a solid block, disk, near net shape, or other shape. Ceramic slurries may be poured into a porous mold (e.g., plaster of Paris or other porous/filtration media) having the desired shape, and cast, for example, under the force of capillary action, vacuum, pressure, or a combination thereof (for example, by methods provided in US 2013/0313738, which is hereby incorporated by reference in its entirety). Green bodies may form a desired shape as water contained in the slurry is absorbed/filtered through the porous media. Excess slurry material, if any remaining, may be poured off the green body. Green bodies removed from molds may dry, for example, at room temperature in a controlled, low humidity environment. Dental milling blanks may be cast, for example, as a solid block, disk or near-net-shape, having dimensions suitable for use in milling or grinding single unit or multi-unit restorations, such as crowns, veneers, bridges, partial or full-arch dentures, and the like.

Manufacturing processes described herein may provide green bodies having relative densities ρ_{R} greater than 48%, such as from 52% to 65% relative density, or such as from 56% to 62% relative density. As used herein, the term “relative density” (β_{R}) refers to the ratio of the measured density ρ_{M} of a sample (g/cm³) to the theoretical density ρ_{T} (3 YSZ—6.083 g/mL; 5 YSZ—6.037 g/mL; 7 YSZ—5.991 g/mL).

Green bodies may be partially consolidated to obtain bisqued bodies by a heating step. Bisquing methods include heating or firing green bodies, such as green bodies in the shape of blocks to obtain, for example, porous bisqued blocks. In some embodiments, relative densities of bisque blocks do not increase more than 5% over the green body density. In some embodiments, the ceramic bodies are bisque heated so that the difference between the relative densities of the bisque body and the green body is 3% or less. Resulting bisqued bodies may be fully dried and have strength sufficient to withstand packaging, shipping, and milling, and in some embodiments, have a hardness value of less than or equal to 0.9 GPa, when tested by the hardness test method described herein. Bisque firing steps may include heating the green body at an oven temperature of from 800° C. to 1100° C. for a holding period of about 0.25 hours to 3 hours, or about 0.25 hours to 24 hours, or by other known bisquing techniques. In some embodiments, bisque processes comprise heating green bodies in an oven heated at an oven temperature of 900° C. to 1000° C. for 30 minutes to 5 hours.

Processes described herein may provide a bisqued body having a relative density ρ_{R} greater than or equal to 48%, such as from 48% to 62%, or from 54% to 60% Bisqued bodies may have a porosity of less than or equal to 45%, such as from 35% to 45%, or from 38% to 42%, or from 38% to 41%. As used herein, the term “porosity”, expressed as percent porosity above, is calculated as: percent porosity=1−percent relative density. A dental block for producing a dental prosthesis includes a zirconia bisqued body having a density of between 56% to 65% of theoretical density and having a porosity of between 35% and 44%, such as between 38% and 41%.

In some embodiments, the median pore size of bisque bodies is less than 200 nm, or less than 150 nm, less than 100 nm, such as from 30 nm to 150 nm, or from 30 nm to 80 nm, or from 35 nm to 40 nm, or from 40 nm to 80 nm, or from 40 nm to 70 nm, or from 45 nm to 75 nm, or from 45 nm to 50 nm, or from 50 nm to 80 nm, or from 50 nm to 75 nm, or from 55 nm to 80 nm, or from 55 nm to 75 nm, when measured according to the methods described herein. As used herein, the term “median pore diameter” refers to the pore diameter measurements obtained from a bisqued body via mercury intrusion performed with an Autopore V porosimeter from Micromeritics Instrument Corp.

Conventional subtractive processes, such as milling or machining processes known to those skilled in the art, may be used to shape a bisqued zirconia ceramic body or milling block into a pre-sintered dental restoration. For dental applications, a pre-sintered restoration may include a dental restoration such as a crown, a multi-unit bridge, an inlay or onlay, a veneer, a full or partial denture, or other dental restoration. For example, bisque stage blocks milled to form bisque-stage dental restorations having anatomical facial surface features including an incisal edge or biting surface, anatomical dental grooves and cusps, and are sintered to densify the bisque-stage restoration into the final dental restoration that may permanently installed in the mouth of a patient. In alternative embodiments, bisque-stage zirconia ceramic bodies are shaped into near-net-shape blocks having generic sizes and shapes that are sintered to theoretical density prior to machining into a final patient-specific dental restoration. The sintered near-net-shape bodies may be prepared having a shape and/or size that is suitable for range of similarly sized and shaped final restoration products.

Dental prostheses may be shaped from porous, pre-sintered blocks by conventional subtractive processes, such as milling or machining processes known to those skilled in the art. The blocks may be shaped in a crown, a multi-unit bridge, an inlay or onlay, a veneer, a full or partial denture, or other dental prosthesis.

After treating bisque stage dental prostheses by applying one or more liquid coloring compositions as disclosed herein, the bisque stage bodies may be “fully sintered” under atmospheric pressure to a density that is at least 98% of the theoretical density of a sintered body. Sintering may occur at oven temperatures in the range of 1200° C. to 1900° C., or 1400° C. to 1600° C., or 1400° C. to 1450° C. Hold times (dwell times) at a temperature within a sintering temperature range may be from 1 minute to 48 hours, such as from 10 minutes to 5 hours, or from 30 minutes to 4 hours, or from 1 hour to 4 hours, or from 1 hour to 3 hours, or from 2 hours to 2.5 hours. Other sintering processes include multi-step sintering processes described in commonly owned U.S. Pat. Pub. 2019/0127284, filed Oct. 31, 2018, hereby incorporated herein by reference in its entirety. Multi-step sintering processes may comprise one or more temperature gradients within a sintering temperature range, with each gradient having the same or different ramp rates, reaching oven temperatures at or above 1200° C., such as from 1200° C. to 1900° C. Multi-step sintering methods may optionally having no hold time within a sintering temperature range, or one hold time or multiple hold times at or above 1200° C. Multi-step sintering processes may have multiple temperature peaks at or above 1200° C., and at least one temperature steps that is between 25° C. to 600° C. lower, or between 50° C. to 400° C. lower, than a preceding or subsequent temperature peak. Hold times at temperature peaks may be between 0 minutes and 30 minutes, and a lower temperature step between two temperature peaks may have a hold time between 2 minutes and 5 hours.

### EXAMPLES

### Example 1

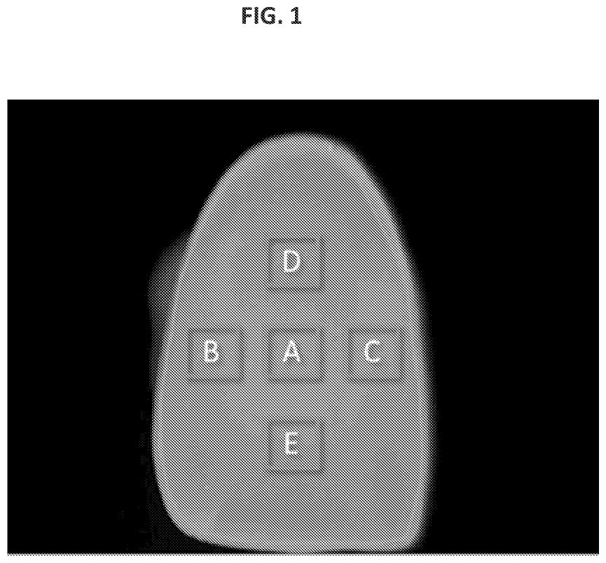

Table 1 lists coloring liquid compositions identified as Solution 1, 2, 3 and 4. ΔE and Ab of Solution 1, 2, 3 and 4 coloring liquid composition-treated yttria stabilized zirconia dental materials compared with the VITA Classical Shade Guide were collected via painting of the composition using a water brush for various Y₂O₃ mol % and repetitive painting times as shown in Table 2. The same painting procedure was utilized for applying Zirkonzahn Color Liquid Prettau© Aquarel compositions for comparison. After drying, the dental material was sintered via a sintering program as shown in Table 3. The ΔE and Ab results are listed in Tables 4 and 5. A smaller ΔE is preferred, indicating a smaller color difference by visual comparison. A smaller Δb is preferred, indicating a smaller difference in the yellowish color. FIG. 1 depicts a crown having locations A, B, C, D and E. The data in Table 4 is for the D location. The data in Table 5 is for the A location. The shade difference for inventive liquid coloring composition-treated 6 mol % Y₂O₃ stabilized ZrO₂ with differing number of painting times is shown in Table 6.

TABLE 1
Composition of Solutions 1, 2, 3 and 4

| | Zn(NO3)2• | AlCl3• | Fe(NO3)3• | Ni(NO3)2• | CuCl2• | MnSO4• | H2O | PPG | FD&C | FD&C |
|---|---|---|---|---|---|---|---|---|---|---|
| | 6H2O | 6H2O | 9H2O | 6H2O | 2H2O | H2O | | 400 | Blue | Yellow |
| Sol 1 | 10.138% | 3.899% | 4.036% | 1.033% | | | 77.982% | 0.971% | | 1.942% |
| Sol 2 | 10.179% | 3.915% | 4.111% | 0.783% | | | 78.101% | 0.971% | | 1.942% |
| Sol 3 | 9.969% | 3.835% | 5.735% | 0.993% | 0.115% | 0.046% | 76.394% | 0.971% | | 1.942% |
| Sol 4 | 9.629% | 3.703% | 7.777% | 1.852% | | 0.059% | 74.067% | 0.971% | 1.942% | |

TABLE 2
Painting Procedure for Y₂O₃ stabilized ZrO₂ dental materials

| Y₂O₃ mol % | Coating per crown | Sintering Program |
|---|---|---|
| 3 | 1 | Prog #1 |
| 4 | 1 | Prog #1 |
| 5 | 2 | Prog #2 |
| 5.5 | 3 | Prog #3 |
| 6 | 3 | Prog #4 |

TABLE 3
Sintering program for Y₂O₃ stabilized ZrO₂ dental materials

| | t1 | T1 | t2 | T2 | t2 | T3 | t4 | T4 | t5 | T5 |
|---|---|---|---|---|---|---|---|---|---|---|
| | (min) | (° C.) | (min | (° C.) | (min) | (° C.) | (min) | (° C.) | (min) | (° C.) |
| Prog#1 | 78 | 1200 | 60 | 1200 | 50 | 1300 | 28 | 1580 | 150 | 1580 |
| Prog#2 | 78 | 1200 | 60 | 1200 | 50 | 1300 | 25 | 1450 | 1 | 1200 |
| Prog#3 | 78 | 1200 | 60 | 1200 | 50 | 1300 | 23 | 1530 | 150 | 1530 |
| Prog#4 | 78 | 1000 | 225 | 1450 | 30 | 1530 | 30 | 1530 | | |
| | t6 | T6 | t7 | T7 | t8 | T8 | t9 | T9 | t10 | T10 |
| | (min) | (° C.) | (min) | (° C.) | (min) | (° C.) | (min) | (° C.) | (min) | (° C.) |
| Prog#1 | | | | | | | | | | |
| Prog#2 | 90 | 1200 | 18 | 1475 | 5 | 1475 | 8 | 1550 | 10 | 1550 |
| Prog#3 | | | | | | | | | | |
| Prog#4 | | | | | | | | | | |

TABLE 4
ΔE and Δb value of coloring liquid treated Y₂O₃ stabilized ZrO₂ dental materials
after sintering at D spot

| | | | ΔE | | | Δb | | |
|---|---|---|---|---|---|---|---|---|
| | | | | Zirkonzahn | | | Zirkonzahn | |
| | | | Zirkonzahn | Color Liquid | | Zirkonzahn | Color Liquid | |
| | | | Color Liquid | Prettau ® | | Color Liquid | Prettau ® | |
| Y2O3 | | | Prettau ® | Aquarell | Invention | Prettau ® | Aquarell | Invention |
| mol % | | Soln | Aquarell | Anterior | Color Liquid | Aquarell | Anterior | Color Liquid |
| 3 | mol % | 1 | 12.5 | 3 | 2.9 | 11.7 | 2.6 | 1.7 |
| | | 2 | 10.5 | 3.9 | 1.7 | 9.9 | 3.9 | 1.3 |
| | | 3 | 10.0 | 2.3 | 1.8 | 7.1 | 0.6 | 0.9 |
| | | 4 | 11.3 | 4.9 | 1.7 | 7.1 | 2.8 | 0.1 |
| 4 | mol % | 1 | 13.5 | 10.6 | 5.9 | 13.0 | 10.2 | 4.9 |
| | | 2 | 13.3 | 9.4 | 3.5 | 13.1 | 9.3 | 2.6 |
| | | 3 | 14.3 | 8.8 | 4.1 | 12.1 | 7.0 | 2.9 |
| | | 4 | 16.6 | 14.4 | 4.9 | 13.1 | 11.2 | 4.0 |
| 5 | mol % | 1 | 12.1 | 8 | 5.4 | 11.6 | 6.8 | 0.6 |
| | | 2 | 13.1 | 14.6 | 4.0 | 13.0 | 13.5 | 1.4 |
| | | 3 | 14.3 | 6.8 | 3.8 | 13.0 | 5.4 | 1.3 |
| | | 4 | 17.0 | 9.5 | 4.1 | 13.5 | 6.9 | 1.5 |
| 5.5 | mol % | 1 | 13.0 | 10.3 | 6.9 | 12.2 | 8.2 | 0.1 |
| | | 2 | 13.1 | 9.3 | 6.9 | 12.7 | 8.6 | 2.8 |
| | | 3 | 12.4 | 8.5 | 10.6 | 11.3 | 7.4 | 5.8 |
| | | 4 | 15.4 | 9.2 | 7.1 | 12.1 | 7.2 | 3.4 |
| 6 | mol % | 1 | 19.9 | 18.3 | 12.4 | 18.1 | 15.8 | 7.4 |

| | | | | | | |
|---|---|---|---|---|---|---|
| 2 | 19.0 | 18.1 | 14.5 | 17.7 | 16.0 | 10.0 |
| 3 | 17.7 | 14.5 | 10.5 | 17.3 | 13.5 | 7.2 |
| 4 | 21.6 | 18.0 | 12.0 | 20.1 | 16.2 | 9.6 |

TABLE 5
ΔE and Δb value of coloring liquid treated Y₂O₃ stabilized ZrO₂ dental materials
after sintering at A spot

| | | | ΔΕ | | | Δb | | |
|---|---|---|---|---|---|---|---|---|
| | | | | Zirkonzahn | | | Zirkonzahn | |
| | | | Zirkonzahn | Color Liquid | | Zirkonzahn | Color Liquid | |
| | | | Color Liquid | Prettau ® | | Color Liquid | Prettau ® | |
| Y₂O₃ | | | Prettau ® | Aquarell | Invention | Prettau ® | Aquarell | Invention |
| mol % | | Soln | Aquarell | Anterior | Color Liquid | Aquarell | Anterior | Color Liquid |
| 3 | mol % | 1 | 14.4 | 3.8 | 2.7 | 13.7 | 3.5 | 1.5 |
| | | 2 | 10.8 | 4.3 | 1.2 | 9.8 | 3.9 | 0.3 |
| | | 3 | 10.3 | 3.2 | 1.7 | 6.7 | 0.4 | 1.2 |
| | | 4 | 13.1 | 6.3 | 2.0 | 7.7 | 2.7 | 0.5 |
| 4 | mol % | 1 | 14.7 | 11.7 | 6.6 | 14.1 | 11.2 | 5.7 |
| | | 2 | 13.7 | 9.5 | 3.1 | 12.9 | 9.3 | 2.4 |
| | | 3 | 14.7 | 9.7 | 4.6 | 12.2 | 7.6 | 3.0 |
| | | 4 | 18 | 15.2 | 4.9 | 13.4 | 11.1 | 3.8 |
| 5 | mol % | 1 | 13.2 | 8.8 | 5.4 | 12.6 | 7.8 | 1.3 |
| | | 2 | 13.8 | 14.6 | 3.2 | 13.4 | 13.7 | 1.7 |
| | | 3 | 15.7 | 7.8 | 3.9 | 14.0 | 6.3 | 1.3 |
| | | 4 | 18.3 | 10.5 | 3.2 | 14.0 | 7.3 | 1.8 |
| 5.5 | mol % | 1 | 14.6 | 11.2 | 6.7 | 13.7 | 9.5 | 1.1 |
| | | 2 | 13.7 | 9.4 | 6.3 | 13.5 | 8.9 | 3.3 |
| | | 3 | 13.5 | 9.2 | 10.9 | 12.2 | 8.0 | 6.4 |
| | | 4 | 16.5 | 10.1 | 5.9 | 12.6 | 7.5 | 3.4 |
| 6 | mol % | 1 | 19.3 | 19.3 | 13.6 | 16.8 | 16.8 | 8.8 |
| | | 2 | 18.8 | 17.7 | 14.6 | 18.0 | 16.1 | 10.8 |

| | | | | | | |
|---|---|---|---|---|---|---|
| 3 | 18.0 | 15.1 | 11.5 | 17.6 | 14.3 | 8.6 |
| 4 | 22 | 18.1 | 11.9 | 20.4 | 16.5 | 10.0 |

TABLE 6
ΔE and Δb of this invention color liquid treated 6
mol % Y₂O₃ stabilized ZrO₂ dental materials at D spot

| | |
|---|---|
| D Spot | A Spot |

| Soln | Coating per crown | ΔE | Δb | ΔE | Δb |
|---|---|---|---|---|---|
| 1 | 6 | 4.5 | 3.2 | 4.2 | 0.1 |
| 2 | 6 | 2.7 | 1.4 | 1.2 | 0.3 |
| 3 | 6 | 5.5 | 3.7 | 5.4 | 3.0 |
| 4 | 6 | 2.9 | 2.4 | 3.4 | 2.9 |

### Example 2

Table 7 lists additional liquid coloring compositions. The compositions were applied to Y₂O₃ stabilized ZrO₂ dental materials. The flexural strength and fracture toughness results are shown in Table 8.

TABLE 7
Comparison of coloring liquid for flexural strength testing

| | Zn(NO3)2• | AlCl3• | Fe(NO3)3• | Ni(NO3)2• | CuCl2• | MnSO4• | Er(NO3)3• |
|---|---|---|---|---|---|---|---|
| | 6H2O | 6H2O | 9H2O | 6H2O | 2H2O | H2O | 6H20 |
| Sol 4 | 9.6% | 3.7% | 7.8% | 1.9% | | 0.1% | |
| Sol 5 | 23.9% | 3.4% | 0.9% | 0.5% | | | |
| Sol 6 | 9.4% | 3.6% | 9.4% | 1.6% | 0.2% | 0.1% | |
| Sol 7 | | 18.2% | 14.6% | 7.2% | 0.7% | 0.1% | 7.3% |
| | Cr(NO3)3• | Tb(NO3)3• | Co(NO3)2• | | PPG | FD&C | FD&C |
| | 9H20 | 6H20 | 6H20 | H2O | 400 | Blue | Yellow |
| Sol 4 | | | | 74.1% | 1.0% | 1.9% | |
| Sol 5 | | | | 68.4% | 1.0% | | 1.9% |
| Sol 6 | | | | 72.9% | 1.0% | 1.9% | |
| Sol 7 | 1.8% | 18.1% | 1.5% | 30.5% | | | |

TABLE 8
Mechanical properties testing of color Liquid
treated different Y₂O₃ mol % treated ZrO₂

| | | | | Fracture |
|---|---|---|---|---|
| Y₂O₃ | Treated | Treatment | Flexural Strength | Toughness |
| mol % | Solution | Times | (MPa) | (MPa*m^{1/2}) |
| 3 | n/a | n/a | 1127 | 5.6 |
| 3 | Sol 7 | 3 | 1085 | 5.4 |
| 6 | n/a | n/a | 600 | 2.2 |
| 6 | Sol 7 | 3 | 616 | 2.2 |
| 5 | Sol 5 | 3 | 994 | 2.8 |
| 5 | Sol 4 | 3 | 925 | 2.8 |
| 5 | Sol 6 | 3 | 923 | 2.8 |

TABLE 9
Precipitants weight percentage of coloring liquid

| | Color | Centrifugation condition | | wt % of |
|---|---|---|---|---|
| | Liquid | RPM | Time | precipitant |
| Zirkonzahn | A2 | 3950 rpm | 5 mins | 0.4% |
| Color Liquid | B2 | 3950 rpm | 5 mins | 2.2% |
| Prettau ® | A4 | 3950 rpm | 5 mins | 0.1% |
| Aquarell | A2 | 5000 rpm | 5 mins | 0.2% |
| | B2 | 5000 rpm | 5 mins | 0.1% |
| | C1 | 5000 rpm | 5 mins | 0.3% |
| | A2 | 6000 rpm | 7 mins | 0.1% |
| | A4 | 6000 rpm | 7 mins | 0.2% |
| | C4 | 6000 rpm | 5 mins | 0.1% |
| This invention | A3.5 | 3950 rpm | 5 mins | 0% |
| Color Liquid | | | | |

### Example 3

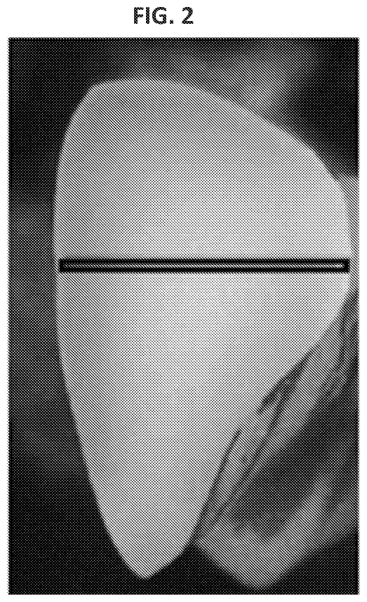

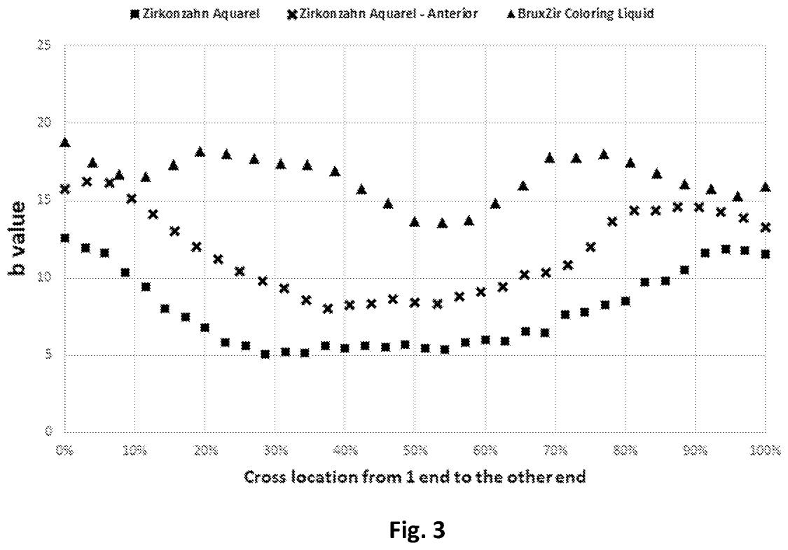

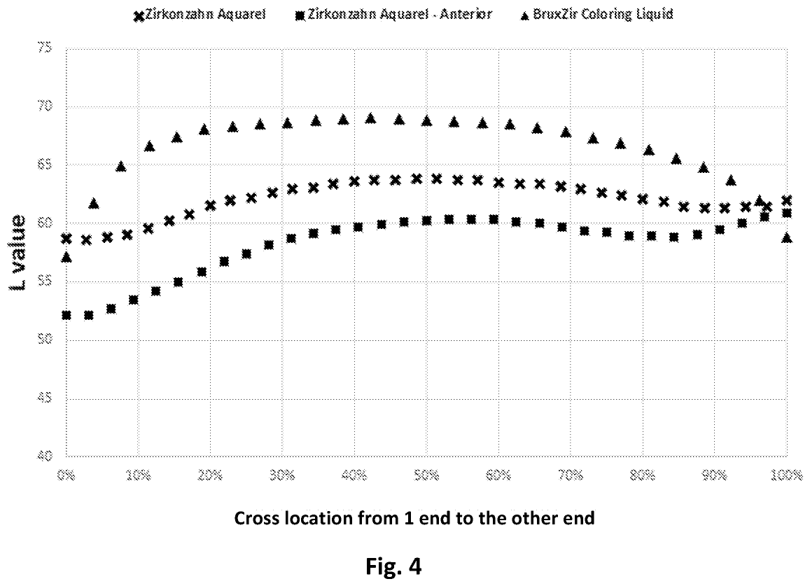

Six coatings of sol 4 from Table 1 were applied to a pontic of 6 mol % Y₂O₃ stabilized ZrO₂. Six coatings of Zirkonzahn Color Liquid Prettau© Aquarel and Zirkonzahn Color Liquid Anterior Prettau© Aquarel compositions, respectively, were applied to a pontic of 6 mol % Y₂O₃ stabilized ZrO₂ for comparison. L and b values were measured on a cross-section of the pontics as shown in FIG. 2. FIGS. 3 and 4 show that the inventive composition provides more chroma and a higher L values and b values.

### Example 4

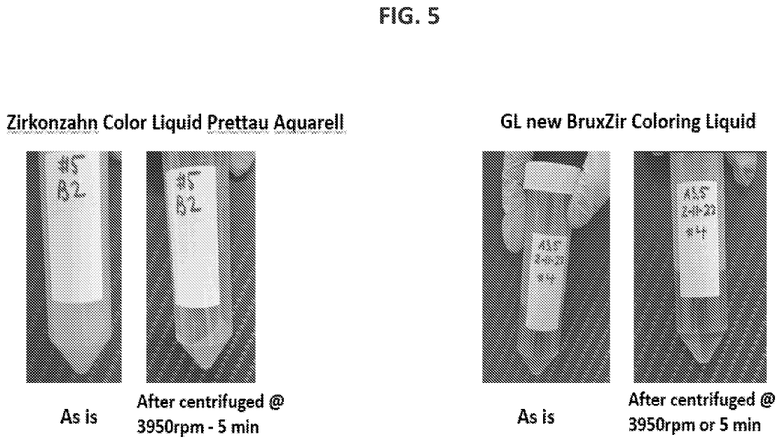

The uniformity of the liquid coloring composition as disclosed herein was compared to Zirkonzahn Color Liquid Prettau Aquarell, as shown in FIG. 5. These coloring liquids were each centrifuged at a speed of 3950 to 6000 rpm to separate all the precipitants from the liquid in the coloring system. After centrifugation, it can clearly be seen that the inventive composition provides a consistent coloring system can without very minimal precipitation. The weight percentage of precipitants of the coloring liquids are listed in Table 9.

### Testing Method:

### Density

For the examples described herein, density calculations of ceramic bodies were determined as follows. The density of green body blocks were calculated by measuring the weight and dividing by the volume calculated from the dimensions of the green block. The density of bisqued body blocks were determined by liquid displacement methods of Archimedes principle. Flat wafers were sectioned or milled from a bisqued block and dried prior to measuring the dry mass. Samples were then saturated with deionized water under vacuum (29-30 in Hg vacuum pressure) for one hour prior to measuring the suspended and saturated masses. All masses were measured to four decimal points precision. A theoretical density was assumed for purposes of calculating relative density of the green body and bisqued body zirconia samples. The term “relative density” (PR) refers to the ratio of the measured density ρ_{M} of a sample (g/cm³) to the theoretical density ρ_{T} (3 YSZ—6.083 g/cm³; 5 YSZ—6.037 g/cm³; 7 YSZ—5.991 g/cm³). For purposes herein, a ceramic material that is fully sintered has a density that is about 98%, or greater, of the theoretical density.

### Translucency

Sintered body translucency was determined by measuring the percent transmittance of D65 light at a wavelength of 700 nm from a 0.95 to 1.05 mm thick sintered sample. Translucency wafers were sectioned or milled from a bisqued block and machined to a diameter corresponding to a final diameter of approximately 30 mm after sinter. The wafers were then ground flat until visually free of defects with 1200 grit and 2000 grit SiC polishing paper. The final bisqued thickness corresponded to 1 mm after sintering and polishing. Samples ground to the desired shape were removed of surface dust and then sintered according to the sintering profile(s) described herein.

After sintering, the samples were polished prior to testing. A polishing procedure was performed using three separate polishing diamond suspensions to remove scratches, 15 μm, 3 μm, and 1 μm, at a rotating speed of 300 rpm for a dwell time of about 5 to 15 minutes, using hand pressure (approximately 2 to 3 pounds).

Total transmittance spectra were measured between the wavelengths of 360 nm to 740 nm with a Konica-Minolta CM5 spectrophotometer illuminated by a D65 light source for all samples. Information contained in the data tables herein refer to measurements at 700 nm or 500 nm wavelengths, as indicated, which are extracted from these measurements. The spectrophotometer was calibrated to white and black prior to measurement. Translucency samples were placed flush against the (approximately) 25 mm integrating sphere aperture. A minimum of two spectra were collected per sample and averaged to yield a final measured transmittance spectra (S-TM). Collected transmittance data may be reported as “percent (%) transmittance”.

### Mercury Porosimetry

Pore size and pore size distributions were measured on a 1 gram to 4 gram sample obtained from a bisqued block. Samples were dried before mercury intrusion. Intrusion was performed with a Micromeritics Autopore V porosimeter with set pressure ranges from total vacuum to 60,000 psi using Micromeritics penetrometers models #07 and #09. The median pore diameter (volume) from the measurement was reported as the Median pore diameter.

### L*a*b Test Along the Cross Section of Pontics for FIG. 2

Spectral image data of sectioned and epoxy-mounted anterior pontics was collected using a SpectroShade Micro II imaging spectrophotometer. Prior collecting spectral image data, the SpectroShade Micro II was calibrated in accordance with built-in calibration instructions provided with the instrument—using the white and green tiles on the docking base provided with the unit. The section face of each specimen was cleaned with isopropyl alcohol and imaged over a dark background (the AC/DC switching adaptor supplied with the SpectroShade Micro II; MEAN WELL ENTERPRISES, GS40A15-P1M). The SpectroShade Micro II (with mouthpiece attached) was then aligned by hand and used to capture a spectral image measurement file for each sample.

SpectroShade measurement files were then uploaded to PC and analyzed using the SpectroShade Analysis software. For each sectioned pontic, CIE L*a*b* color space value sets were extracted from programmatically-selected, approximately 0.17×0.17 mm square areas adjacent to one another across the gingiva region from the approximate labial edge to the approximate lingual edge of each sample face using the SpectroShade Analysis software. With display resolution set to 1680×1050, programmatic selection of areas was performed using AutoHotKey desktop automation software to incrementally select 10×10 pixel areas (corresponding to areas of approximately 0.17×0.17 mm) within the SpectroShade Analysis software, extract the L*a*b color space values for the selected area to clipboard with a call to Capture2Text optical character recognition software, paste the L*a*b values into a Notepad document, and repeat the process for the adjacent area—starting from a point at the labial edge, passing through a greater portion of the gingival half of the pontic, and ending at the lingual edge.

### L*a*b Test for FIG. 1

Spectral image data of the labial faces of glazed crowns was collected using a SpectroShade Micro II imaging spectrophotometer. Prior collecting spectral image data, the SpectroShade Micro II was calibrated in accordance with built-in calibration instructions provided with the instrument—using the white and green tiles on the docking base provided with the unit. Crowns were imaged over a dark background (the AC/DC switching adaptor supplied with the SpectroShade Micro II; MEAN WELL ENTERPRISES, GS40A15-P1M). A small dot of wax was used to support the crown by the cingulum upon the dark background such that the labial face was approximately level with the dark background surface and exposed for spectral imaging. The SpectroShade Micro II (with mouthpiece attached) was then aligned by hand and used to capture a spectral image measurement file for each crown.

SpectroShade measurement files were then uploaded to PC and analyzed using the SpectroShade Analysis software. For each crown, L*a*b color space value averages were collected from seven areas (titled A through E). Areas A through E were each set with a cursor size setting of 50 within the SpectroShade Analysis software (corresponding to an area of 1.6×1.6 mm on the crown face) and correspond to approximate center, left center, right center, upper center, and lower center regions (FIG. 1). AutoHotKey desktop automation software was utilized to incrementally select 10×4 pixel areas (corresponding to areas of approximately 180×72 microns) within the SpectroShade Analysis software, extract the L*a*b color space values for the selected area to clipboard with a call to Capture2Text optical character recognition software, paste the L*a*b values into a Notepad document, and repeat the process for the targeted area.

In view of the many possible embodiments to which the principles of the disclosed invention may be applied, it should be recognized that the illustrated embodiments are only preferred examples of the invention and should not be taken as limiting the scope of the invention.

What is claimed is:

1. A method for coloring a porous ceramic dental body comprising:
applying at least one coating of a liquid coloring composition to at least a portion of a surface area of the porous ceramic dental body, the liquid coloring composition comprising:
(a) at least one opaquing agent comprising a material comprising Zn, Al, Si, or a combination thereof;
(b) at least one coloring agent comprising a material comprising Fe, Ni, Cu, Mn, Co, Cr, Mo, Pr, Nd, Er, Ce, Tb, or a combination thereof,
(c) at least one wetting agent; and
(d) at least one solvent; and
sintering the coated porous ceramic dental body to obtain a fully sintered ceramic body having a density that is at least 98% of the theoretical density.

2. The method of claim 1, wherein the coloring matches natural teeth.

3. The method of claim 1, wherein the coloring matches a shade from a VITA A1-D4® Classical Shades shade guide or a VITA Bleached Shades shade guide.

4. The method of claim 1, wherein the sintered ceramic dental body has a transmittance of 40% to 80% at 700 nm (when measured on a 1 mm thick fully sintered ceramic body).

5. The method of claim 1, wherein the sintered ceramic dental body has a transmittance of 50% to 70% at 700 nm (when measured on a 1 mm thick fully sintered ceramic body).

6. The method of claim 1, wherein the porous ceramic dental body comprises yttria-stabilized zirconia ceramic, stabilized by 2 mol % to 10 mol % yttria.

7. The method of claim 1, wherein the porous ceramic dental body comprises yttria-stabilized zirconia ceramic, stabilized by 3 mol % to 6.5 mol % yttria.

8. The method of claim 1, wherein the opaquing agent is selected from a Zn(NO₃)₂·6H₂O, AlCl₃·6H₂O, tetraethyl orthosilicate, or a mixture thereof.

9. The method of claim 1, wherein the liquid coloring composition comprises a Zn(NO₃)₂·6H₂O opaquing agent and an AlCl₃·6H₂O opaquing agent.

10. The method of claim 9, wherein the liquid coloring composition comprises 1 wt % to 30 wt % Zn(NO₃)₂·6H₂O, and 1 wt % to 10 wt % AlCl₃·6H₂O, based on the total weight of the composition.

11. The method of claim 9, wherein the weight ratio of Zn(NO₃)₂·6H₂O/AlCl₃·6H₂O is in the range of 1 to 10.

12. The method of claim 1, wherein the liquid coloring composition comprises 9 wt % to 11 wt % of a Zn-containing opaquing agent, and 3 wt % to 4 wt % of an Al-containing opaquing agent, based on the total weight of the composition.

13. The method of claim 1, wherein the liquid coloring composition comprises 22 wt % to 25 wt % of a Zn-containing opaquing agent, and 3 wt % to 4 wt % of an Al-containing opaquing agent, based on the total weight of the composition.

14. The method of claim 1, wherein the liquid coloring composition comprises a Zn-containing and an Al-containing opaquing agent, and the weight ratio of Zn metal ion/Al metal ion is in the range of 3 to 15.

15. The method of claim 14, wherein the coloring agent is selected from Fe(NO₃)₃·9H₂O, Ni(NO₃)₂·6H₂O, CuCl₂·2H₂O, MnSO₄·H₂O, Er(NO₃)₃·6H₂O, CoCl₂·6H₂O, Nd(NO₃)₃·6H₂O, Cr(NO₃)₃·9H₂O, Tb(NO₃)₃·6H₂O, Pr(NO₃)₃·6H₂O, or a mixture thereof.

16. The method of claim 14, wherein the liquid coloring composition comprises an Fe(NO₃)₃·9H₂O coloring agent and a Ni(NO₃)₂·6H₂O coloring agent.

17. A liquid coloring composition comprising:
(a) a Zn(NO₃)₂·6H₂O opaquing agent;
(b) an AlCl₃·6H₂O opaquing agent;
(c) an Fe(NO₃)₃·9H₂O coloring agent;
(d) a Ni(NO₃)₂·6H₂O coloring agent;
(e) polypropylene glycol; and
(g) at least one solvent.

18. The composition of claim 17, comprising 1 wt % to 30 wt % Zn(NO₃)₂·6H₂O, and 1 wt % to 10 wt % AlCl₃·6H₂O, based on the total weight of the composition.

19. A coloring system kit for coloring a porous ceramic dental body comprising at least 20 unique liquid coloring compositions, wherein each coloring composition comprises:
(a) a Zn(NO₃)₂·6H₂O opaquing agent;
(b) an AlCl₃·6H₂O opaquing agent;
(c) an Fe(NO₃)₃·9H₂O coloring agent;
(d) a Ni(NO₃)₂·6H₂O coloring agent;
(e) polypropylene glycol; and
(g) at least one solvent.

20. The coloring system kit of claim 19, comprising 26 unique liquid coloring compositions.
